(19) United States
(12) Patent Application Publication
(10) Pub. No.: US 2004/0176381 A1
(43) Pub. Date: Sep. 9, 2004

(54) Methotrexate compliance packaging

(76) Inventor: Michael J. Walsh, San Diego, CA (US)

Correspondence Address: Cathryn Campbell, McDERMOTT, WILL & EMERY, 7th Floor, 4370 La Jolla Village Drive, San Diego, CA, 92122, US

(21) Appl. No.: 10/384,353

(22) Filed: Mar. 6, 2003

(51) Int. Cl. B65D 83/04; B65D 85/42; A61K 31/525

(52) U.S. Cl. 514/251; 206/538

(57) ABSTRACT

The present invention provides a compliance system that contains at least one dispenser including at least one individual dose of an anti-folate therapeutic and at least one individual dose of a folic acid analog, each individual dose of anti-folate therapeutic and folic acid analog positioned in one or more individual compartments. Such a dispenser can have, for example, suitable indicia marked in association with each individual compartment, thereby identifying each compartment with the day or time when the enclosed anti-folate therapeutic or folic acid analog should be administered.

### BACKGROUND OF THE INVENTION

1. Field of the Invention

This invention relates generally to anti-folate drug therapy and, more specifically, to compliance packaging for anti-folate therapeutics such as methotrexate in combination with folic acid supplements.

2. Background Information

Folate (folic acid) is a vitamin that is essential for the life-sustaining processes of DNA synthesis, replication and repair. Folate is also important for protein biosynthesis, another process that is central to cell viability. The pteridine compound, methotrexate, is structurally similar to folate and, as a result, can bind to the active sites of a number of enzymes that normally use folate as a coenzyme for biosynthesis of purine and pyrimidine nucleotide precursors and for the interconversion of amino acids during protein biosynthesis. Despite its structural similarity to folic acid, methotrexate cannot be used as a cofactor by enzymes that require folate and instead competes with the folate cofactor for enzyme binding sites, thereby inhibiting protein and DNA biosynthesis and, hence, cell division.

Methotrexate has been exploited in the treatment of a number of diseases and conditions that are characterized by rapid or aberrant cell growth. As an example, autoimmune diseases are characterized by an inappropriate immune response directed against normal autologous (self) tissues and are mediated by rapidly replicating T-cells or B-cells. Autoimmune diseases as well as asthma and graft-versus-host conditions have been treated with methotrexate including, without limitation, conditions such as rheumatoid arthritis, psoriasis, multiple sclerosis, the autoimmune stage of Type 1 diabetes, systemic lupus erythematosus, autoimmune uveoretinitis, myasthenia gravis and autoimmune thyroiditis.

Despite its therapeutic efficacy, treatment with methotrexate can present a risk to the patient. In particular, methotrexate therapy can cause a variety of adverse side effects that mimic folate deficiency including, for example, cytopenia, gastrointestinal intolerance, stomatitis and alopecia. This situation is especially problematic in the treatment of chronic conditions such as rheumatoid arthritis, where methotrexate can be administered over a period of many years.

In the case of anti-folate therapeutics such as methotrexate, taking the prescribed dose at the prescribed time is crucially important. The toxicity of methotrexate therapy is dose dependent, and fatalities have resulted when patients have mistakenly taken their prescribed weekly dose of methotrexate on a daily basis. Compliance with methotrexate regimens is further complicated by the additional prescription of compensatory doses of folic acid supplements, which can alleviate the side-effects of methotrexate therapy.

Thus, there exists a need for compliance packaging of methotrexate or another anti-folate therapeutic together with a folic acid analog. The present invention satisfies this need and provides related advantages as well.

### SUMMARY OF THE INVENTION

The present invention provides a compliance system that contains at least one dispenser including at least one individual dose of an anti-folate therapeutic and at least one individual dose of a folic acid analog, each individual dose of anti-folate therapeutic and folic acid analog positioned in one or more individual compartments. Such a dispenser can have, for example, suitable indicia marked in association with each individual compartment, thereby identifying each compartment with the day or time when the enclosed anti-folate therapeutic or folic acid analog should be administered.

In a compliance system of the invention, the dispenser can include, for example, at least one individual dose of the anti-folate therapeutic and at least five daily individual doses of the folic acid analog. A dispenser included in a compliance system of the invention also can include, for example, at least one individual dose of the anti-folate therapeutic and exactly six daily individual doses of the folic acid analog.

A variety of anti-folate therapeutics are useful in a compliance system of the invention including, without limitation, methotrexate and analogs thereof. In particular embodiments, a dispenser includes exactly one weekly dose of anti-folate therapeutic such as methotrexate. In such compliance systems, the weekly dose of methotrexate can be, for example, in the range of 2.5 mg to 40 mg such as, without limitation, a weekly dose of 2.5 mg, 5 mg, 7.5 mg, 10 mg, 15 mg, 20 mg, 25 mg, or higher.

In one embodiment, a compliance system of the invention includes a 2.5 mg weekly dose of methotrexate. Such a weekly dose can be included in the dispenser as, for example, a single individual dose.

In another embodiment, a compliance system of the invention includes a 5 mg weekly dose of methotrexate. Such a weekly dose can be included in the dispenser as, for example, a single individual dose.

In a further embodiment, a compliance system of the invention includes a 7.5 mg weekly dose of methotrexate. Such a weekly dose can be included in the dispenser as, for example, a single individual dose or can be separated into two or more individual doses such as three individual doses of 2.5 mg.

In yet another embodiment, a compliance system of the invention includes a 10 mg weekly dose of methotrexate. Such a weekly dose can be included in the dispenser as, for example, a single individual dose or can be separated into two or more individual doses.

In an additional embodiment, a compliance system of the invention includes a 15 mg weekly dose of methotrexate. Such a weekly dose can be included in the dispenser as, for example, a single individual dose or can be separated into two or more individual doses.

A variety of folic acid analogs can be useful in the compliance systems of the invention including, without limitation, folic acid and folinic acid. In one embodiment, a compliance system of the invention contains a dispenser which includes a daily individual dose of folinic acid in the range of 0.5 to 2 mg. In additional embodiments, a compliance system of the invention contains a dispenser which includes a daily individual dose of folinic acid which is 1 mg or 2 mg.

In a dispenser included in a compliance system of the invention, the individual compartments can be, for example, arrayed linearly. A compliance system of the invention also can include at least two dispensers and can further include exactly four dispensers. A compliance system of the invention also can optionally include, for example, a patient information insert and can be packaged, if desired, in an outer container. In a compliance system of the invention, a dispenser can optionally include, if desired, a visual or audio alarm or a means for recording when individual compartments are opened.

The present invention further provides a compliance system that contains four dispensers, each including at least one individual dose of an anti-folate therapeutic and at least five daily individual doses of a folic acid analog, where each individual dose of anti-folate therapeutic or folic acid analog is positioned in one or more individual compartments and where each dispenser has suitable indicia marked in association with each individual compartment, thereby identifying each compartment with the day or time when the enclosed anti-folate therapeutic or folic acid analog should be administered.

Further provided herein is a compliance system that contains at least one dispenser which includes exactly four weekly doses of anti-folate therapeutic and exactly four weekly doses of folic acid analog, where each of the weekly doses of anti-folate therapeutic or folic acid analog is divided into individual doses sequentially arrayed in the dispenser.

### BRIEF DESCRIPTION OF THE DRAWINGS

FIG. 1 shows the chemical structures of exemplary anti-folate therapeutics and folic acid analogs. (A) The chemical structure of methotrexate. (B) The chemical structure of folic acid. (C) The chemical structure of folinic acid.

FIG. 2 shows various exemplary dispensers. Each dispenser is shown as a rectangle. Individual compartments are shown are circles. “MTX” represents methotrexate, and “FA” represents folic acid. Compartments containing methotrexate are shaded black while compartments containing folic acid are shaded gray and compartments with placebo are unshaded. Doses are indicated in milligrams under the particular medicament. (A) Dispenser with a weekly dose of 2.5 mg methotrexate administered as a single individual dose, with daily individual doses of 2 mg folic acid given every day except when methotrexate is taken. (B) Dispenser with a weekly dose of 7.5 mg methotrexate administered as a single individual dose made up of three 2.5 mg tablets, with daily individual doses of 1 mg folic acid given five days of the week. (C) Dispenser with a weekly dose of 7.5 mg methotrexate administered as three individual doses of 2.5 mg, with daily individual doses of 1 mg folic acid given five days of the week, on the days when methotrexate is not given. (D) Dispenser with a weekly dose of 15 mg methotrexate administered as three individual doses of 5 mg given over a 24 hour period, with daily individual doses of 2 mg folic acid given five days of the week, on days when methotrexate is not given. (E) Dispenser with a weekly dose of 7 mg methotrexate administered as twice daily individual doses of 0.5 mg every day of the week, with daily individual doses of 1 mg folic acid every day of the week. (F) Calendar-style dispenser with a month's worth of medication. A weekly dose of 5 mg methotrexate is administered as a single individual dose and as a single tablet, with individual doses of 2 mg folic acid given six days of the week.

### DETAILED DESCRIPTION OF THE INVENTION

Pharmaceutical non-compliance is a tremendous economic and medical problem. Some analyses indicate that, of all medications prescribed, less than 70 percent are actually consumed. Furthermore, as many as 40 percent of patients receiving outpatient drug therapy experience a treatment failure or new medical problem as a result of non-compliance. In the United States, pharmaceutical non-compliance drains an estimated $100 billion from the national economy and may account for the deaths of over 125,000 Americans annually, which equates to more than 300 people every day. In addition, ten percent of all hospital admissions are the result of pharmaceutical non-compliance, while more than twenty percent of all nursing home admissions are due to the inability of patients to take their medications as prescribed.

Patient compliance has been defined as “the extent to which an individual's behavior coincides with medical or health advice.” (Remington's Pharmaceutical Sciences Chapter 103, Volume II, page 1796 (19^{th} Edition (1995)). Conversely, non-compliance encompasses a variety of behaviors including drug underuse, which encompasses taking too low a dose or skipping a dose. Non-compliance also encompasses drug overuse such as taking too high a dose or taking a dose too frequently. Medication compliance is effected by the physician's and pharmacist's relationship with the patient, and, in particular, how clearly the physician or pharmacist explains the treatment regimen to the patient. Non-compliance is generally higher in the elderly population than in other groups; for patients over the age of 65, about 20% of all non-elective hospital admissions are due to mismanagement of prescription medications. The increased incidence of non-compliance in the elderly population may be due, for example, to declining mental function, increasing numbers of medications prescribed or an increase in side effects or drug interactions associated with multiple drug regimens (Murray et al., DICP 20:146 (1986)). Unfortunately, counseling, education and behavior modification techniques have achieved only limited success in boosting patient compliance.

Pharmaceutical non-compliance is a particularly urgent problem in the case of methotrexate, a drug which can be fatal when ingested at excessive doses. The parent compound of methotrexate, aminopterin, has been used to treat patients with rheumatoid arthritis, psoriasis, and psoriatic arthritis since the 1950s. Low dose weekly methotrexate was first used to treat patients with active rheumatoid arthritis in the 1970s. Over the years, methotrexate has become the most widely used agent among disease modifying anti-rheumatic drugs (DMARD) and has been ranked ahead of such drugs in terms of its efficacy/toxicity ratio (O'Dell, Rheum. Dis. Clin. North Am. 23:779-796 (1997)). In particular, one-third of rheumatoid arthritis patients show major improvement on methotrexate, with this drug generally preferred over azathioprine, sulfasalazine, gold salts and penicillamine because of its relatively favorable ratio of efficacy to toxicity (Felson et al., Arthritis Rheum. 35:1117-1125 (1992); Maetzel et al., Rheum. 39:975-981 (2000); and Alarcón et al., J. Rheum. 19:1868-1873 (1992)). In some cases, methotrexate is combined with other disease modifying anti-rheumatic drugs such as sulfasalazine and hydroxychloroquine or other anti-inflammatory agents, for example, anti-cytokine therapeutics such as anti-tumor necrosis factor-α antibodies (O'Dell, Rheum. Dis. Clin. North Am. 24:465-477 (1998); Kremer et al., Rheum. Dis. Clin. North Am. 24:651-658 (1998); and O'Dell and Scott, Rheum. 38 Suppl. 2:24-26 (1999)).

Most patients demonstrate a dose dependent response to methotrexate, which is generally administered weekly (Furst et al., J. Rheum. 16:313-320 (1989)). Adverse effects are also dose-dependent, and adverse effects, rather than lack of efficacy, are the most common reason for discontinuing methotrexate therapy (Alarcon et al., Arthritis Rheum. 32:671-676 (1989)). In rheumatoid arthritis patients on methotrexate, mild adverse effects occur in up to 60% patients, with roughly 7 to 30% of patients discontinuing therapy within the first year of treatment (Schnabel and Gross, Sem. Arthritis Rheum. 23:310-327 (1994); Kremer and Phelps, Arth. Rheum. 35:138-145 (1992)). Gastrointestinal intolerance such as nausea, abdominal pain, indigestion or diarrhea; asymptomatic elevation of serum hepatic transaminase levels; and stomatitis are the major reasons for dose reduction or premature discontinuation of methotrexate therapy (Kremer, Scand. J. Rheum. 25:341-344 (1996); Morgan et al., Arth. Rheum. 0.30:1348-1356 (1987); Andersen et al., J. Rheum. 24:830-837 (1997); Leeb et al., Clin. Exp. Rheum. 13:459-463 (1995); and Dijkmans, J. Rheum. 34:1172-1174 (1995)). In addition to dose and duration of treatment, other factors such as folate deficiency, advanced age, cumulative dose, renal insufficiency and concomitant use of other anti-folates can influence methotrexate toxicity (Wallace and Sherry, J. Rheum. 22:1009-1112 (1995); and Jackson, Pharm. Ther. 25:61-82 (1984)).

Many adverse effects such as gastrointestinal intolerance, stomatitis, alopecia and cytopenia mimic folate deficiency and can be explained by the antifolate properties of methotrexate (Bannwarth et al., Drugs 47:25-50 (1994); Van Ede et al., Sem. Arthr. Rheum. 27:277-292 (1998); and Segal et al., Sem. Arthr. Rheum. 20:190-200 (1990)). Depleted intracellular folate levels have been documented in hepatocytes and peripheral blood lymphocytes of methotrexate-treated patients (Stenger et al., Ann. Rheum. Dis. 51:1019-1020 (1992); Morgan et al., Clin. Pharm. Ther. 50:547-556 (1991); Kremer et al., Arth. Rheum. 29:832-834 (1986); Leeb et al., supra, 1995; Morgan et al., Arth. Rheum. 30:1348-1356 (1987); Hine et al., Arth. Rheum. 33 (Suppl.): S60 (1990); and Stewart et al., Sem. Arth. Rheum. 331:906-908 (1988)). Folate deficiency occurs frequently in patients with rheumatoid arthritis, and folate stores are further decreased in rheumatoid arthritis patients taking methotrexate (Leeb et al., supra, 1995). Several studies have shown the advantages of folic or folinic acid supplementation in rheumatoid arthritis and other patients undergoing treatment with methotrexate (Ortiz et al., J. Rheum. 25:36-43 (1998)); Kremer et al., supra, 1996; Dijkmans, supra, 1995; Bannwarth et al., supra, 1994; Van Ede et al., supra, 1998; Segal et al., supra, 1990; Cronstein, Arthr. Rheum. 39:1951-1960 (1996); Endresen and Husby, Scand. J. Rheum. 30:129-134 (2001); Griffith et al., Rheum. 39:1102-1109 (2000); and van Ede et al., Arth. Rheum. 44:1515-1524 (2001)). As an example, in double-blind studies, 5 mg of folic acid or 2.5 to 5 mg per week of folinic acid, an activated form of folic acid, substantially reduced side effects of methotrexate without interfering with therapeutic efficacy in rheumatoid arthritis patients (Morgan et al., Ann. Intern. Med. 121:833-841 (1994); and Shiroky et al., Arthr. Rheum. 36:795 (1993)). Similarly, 5 mg per day folic acid was shown to alleviate the side effects from methotrexate observed in patients with severe psoriasis (Duhra, J. Am. Acad. Dermatol. 28:466-469 (1993)). The folic or folinic acid was generally prescribed to be taken at a different time from methotrexate and, in some cases, was prescribed to be taken only five days per week. These results demonstrate the advantage of a regimen that combines a folic acid analog with an anti-folate therapeutic such as methotrexate.

The present invention provides a compliance system useful for home administration of an anti-folate therapeutic such as low-dose methotrexate in combination with one or more folic acid analogs. In particular, the invention provides a compliance system that contains at least one dispenser including at least one individual dose of an anti-folate therapeutic and at least one individual dose of a folic acid analog, each individual dose of anti-folate therapeutic and folic acid analog positioned in one or more individual compartments. Such a dispenser can have, for example, suitable indicia marked in association with each individual compartment, thereby identifying each compartment with the day or time when the enclosed anti-folate therapeutic or folic acid analog should be administered.

In a compliance system of the invention, the dispenser can include, for example, at least one individual dose of anti-folate therapeutic and at least five daily individual doses of folic acid analog. A dispenser included in a compliance system of the invention also can include, for example, at least one individual dose of anti-folate therapeutic and exactly six daily individual doses of folic acid analog.

It is understood that the compliance systems of the invention can be useful for any patient prescribed methotrexate or other anti-folate therapeutic including, but not limited to, any outpatient prescribed methotrexate or other anti-folate therapeutic that is associated with side effects due to folate deficiency. One skilled in the art understands that a compliance system of the invention, can be useful for patients suffering from any of a variety of disorders including, but not limited to, rheumatoid and other forms of arthritis, psoriasis, Crohn's disease, ulcerative colitis, systemic lupus erythematosus, systemic vasculitis, polymiositis, multiple sclerosis, the autoimmune stage of Type 1 diabetes, autoimmune uveoretinitis, myasthenia gravis, autoimmune thyroiditis, asthma, and graft-versus-host disease (Alarcón, Immunopharm. 47:259-271 (2000), and Sato, Lupus 10:162-164 (2001)).

A compliance system of the invention contains at least one dispenser that includes at least one individual dose of an anti-folate therapeutic and at least one individual dose of a folic acid analog, each individual dose of anti-folate therapeutic and folic acid analog positioned in one or more individual compartments. Such a compliance system can have, for example, a single dispenser that includes exactly one weekly dose of anti-folate therapeutic such as methotrexate.

Methotrexate and other anti-folate therapeutics are typically prescribed as a weekly dose to be administered as a single individual dose or two or three individual doses administered within 24-36 hours, for example, two doses administered 12 hours apart. Folic acid analogs are typically administered as a single dose either once daily, six days a week, or five days a week. It has been shown that folic acid can be taken together with methotrexate without altering the efficacy of the methotrexate (Alarcón, supra, 2000).

The weekly dose of an anti-folate therapeutic included in a compliance system of the invention is provided in an effective amount. Such an amount generally is the minimum dose necessary to achieve the desired reduction in severity of one or more symptoms of the condition to be treated, for example, arthritis, psoriasis, Crohn's disease, ulcerative colitis or systemic lupus erythematosus, such as that amount roughly necessary to reduce the discomfort caused by the condition to tolerable levels or to result in a significant reduction in the discomfort caused by the condition. Such amounts generally are in the range of 0.1-1000 mg/week and can be, for example, in the range of 0.1-500 mg/week, 0.5-500 mg/week, 0.5-100 mg/week, 0.5-50 mg/week, 0.5-30 mg/week, 1-20 mg/week, 2.5-20 mg/week or 2.5-15 mg/week, with the actual amount to be prescribed and included in the compliance system determined by a physician taking into account the relevant circumstances including the severity and type of condition to be treated, the age and weight of the patient, the patient's general physical condition, the cumulative dose, the characteristics of the active compounds and pharmaceutical formulation, and the route or routes of administration.

As used herein in reference to an anti-folate therapeutic or folic acid analog, the term “weekly dose” means the total amount of anti-folate therapeutic prescribed to be taken within one week (seven days). It is understood that a weekly dose can be prescribed to be administered, for example, as a single individual dose, or can be prescribed to be administered in two, three, or more individual doses, or can be prescribed to be administered in daily, twice daily or thrice daily individual doses to be taken every day of the week. Thus, where, within a single week, 7.5 mg methotrexate is prescribed to be administered as a single individual dose, the weekly dose of methotrexate is 7.5 mg. Similarly, where, within a single week, 5 mg is prescribed to be administered one morning, followed by 5 mg administered the same evening, and 5 mg administered again the next morning, the weekly dose of methotrexate is 15 mg. It is understood that, where a weekly dose is administered as a single dose, the weekly dose will be the same as the individual dose, defined herein below.

Weekly doses of methotrexate include, but are not limited to, weekly doses of 1 to 50 mg such as weekly doses of 2.5 to 40 mg. As non-limiting examples, a weekly dose of methotrexate can be 1 mg, 2.5 mg, 3.5 mg, mg, 7 mg, 7.5 mg, 10 mg, 14 mg, 15 mg, 17.5 mg, 20 mg, 21 mg, 24.5 mg, 25 mg, 30 mg, 35 mg, 40 mg, 45 mg, 49 mg, 50 mg, 60 mg, 70 mg, 80 mg, 90 mg or 100 mg. Any of such weekly doses can be included in the dispenser as, for example, a single individual dose, two or more individual doses, daily individual doses to be administered every day of the week, or twice daily individual doses to be administered every day of the week. In particular embodiments, any of such weekly doses are included in the dispenser as daily individual doses to be administered every day of the week. In further embodiments, any of such weekly doses are included in the dispenser as twice daily individual doses to be administered every day of the week, or as thrice daily individual doses to be administered every day of the week. As one non-limiting example, a 3.5 mg weekly dose of methotrexate or another anti-folate therapeutic can be included in the dispenser as seven daily individual doses of 0.5 mg. As another non-limiting example, a 7 mg weekly dose of methotrexate or another anti-folate therapeutic can be included in the dispenser as seven daily individual doses of 1 mg.

Weekly doses of folic acid or folinic acid include, without limitation, doses in the range of about 1 to 40 mg, for example, 2.5 to 30 mg. Such a weekly dose of folic or folinic acid can be, without limitation, 2.5 mg, 3 mg, 3.5 mg, 5 mg, 6 mg, 7 mg, 10 mg, 12 mg, 14 mg, 15 mg, 18 mg, 20 mg, 21 mg, 24 mg, 25 mg, 27.5 mg, 28 mg, 30 mg, 35 mg, 40 mg, 45 mg, 50 mg, 55 mg, 60 mg, 65 mg, 70 mg, 75 mg, 80 mg or more. As non-limiting examples, a 2.5 mg, 3 mg or 3.5 mg weekly dose of folic or folinic acid can be administered as 5, 6 or 7 daily individual doses of 0.5 mg; a 5, 6 or 7 mg weekly dose of folic or folinic acid can be administered as 5, 6 or 7 daily individual doses of 1 mg; a 10, 12 or 14 mg weekly dose of folic or folinic acid can be administered as 5, 6 or 7 daily individual doses of 2 mg or as 5, 6 or 7 twice daily individual doses of 1 mg; a 15, 18 or 21 mg weekly dose of folic or folinic acid can be administered as 5, 6 or 7 daily individual doses of 3 mg; a 20, 24 or 28 mg weekly dose of folic or folinic acid can be administered as 5, 6 or 7 daily individual doses of 4 mg; and a 25, 30 or 35 mg weekly dose of folic or folinic acid can be administered as 5, 6 or 7 daily individual doses of 5 mg, etc.

Weekly doses of an anti-folate therapeutic such as methotrexate or a folic acid analog such as folic or folinic acid can be expressed as a ratio, for example, a ratio of 0.25 to 1; 0.5 to 1; 1 to 1; 1 to 1.5; 1 to 2; 1 to 3; 1 to 4; 1 to 5; or 1 to 10 milligrams of an anti-folate therapeutic such as methotrexate to milligrams of a folic acid analog such as folic or folinic acid. As non-limiting examples, a dispenser can include a 3.5 mg weekly dose of methotrexate and a 3.5 mg weekly dose of folic or folinic acid; a 3.5 mg weekly dose of methotrexate and a 7 mg weekly dose of folic or folinic acid; a 7 mg weekly dose of methotrexate and a 7 mg weekly dose of folic or folinic acid; a 7 mg weekly dose of methotrexate and a 14 mg weekly dose of folic or folinic acid; a 7 mg weekly dose of methotrexate and a 21 mg weekly dose of folic or folinic acid; a 7 mg weekly dose of methotrexate and a 28 mg weekly dose of folic or folinic acid; a 14 mg weekly dose of methotrexate and a 7 mg weekly dose of folic or folinic acid; a 14 mg weekly dose of methotrexate and a 14 mg weekly dose of folic or folinic acid; or a 14 mg weekly dose of methotrexate and a 28 mg weekly dose of folic or folinic acid. Such weekly doses of methotrexate and folic or folinic acid can be, without limitation, administered as seven daily individual doses or in other regimens. One skilled in the art understands that these and other ratios of weekly doses of an anti-folate therapeutic to a folic acid analog are encompassed by the compliance systems of the invention.

As used herein in reference to an anti-folate therapeutic or folic acid analog, the term “individual dose” means the total amount of therapeutic and analog prescribed to be administered at a particular time, for example, on a particular day or particular hour of a particular day. Thus, an individual dose is defined by the time at which it is prescribed to be taken; such a dose can be provided, for example, as a single pill or multiple pills, which can be packaged together in the same compartment or packaged in two or more individual compartments, provided that the multiple pills are prescribed to be taken by the patient at the same time. In view of the above, it is understood that an individual dose can be composed of a single pill, tablet, capsule, spoonful, vial, ampule etc., or can be composed of multiple pills, tablets, capsules, spoonfuls, vials, ampules, etc., or a combination thereof. Two different individual doses are typically prescribed to be administered at two times separated by two or more hours such as, without limitation, four hours, eight hours, 12 hours, 24 hours or more.

Individual doses of methotrexate or another anti-folate therapeutic include, but are not limited to, individual doses of 0.1 mg, 0.2 mg, 0.3 mg, 0.4 mg, 0.5 mg, 0.6 mg, 0.7 mg, 0.8 mg, 0.9 mg, 1.0 mg, 1.5 mg, 2.0 mg, 2.5 mg, 5.0 mg, 7.5 mg, and 10 mg. In specific embodiments, such an individual dose is administered one daily, twice daily or thrice daily.

The term “daily individual dose,” as used herein in reference to a dose of an anti-folate therapeutic or folic acid analog, means an individual dose prescribed to be taken once a day. A daily individual dose is typically prescribed to be taken for several days in a row and can be prescribed to be taken at the same time of day.

As a non-limiting example, an individual dose of 7.5 mg methotrexate can be provided as a single 7.5 mg tablet in a single compartment, or as three 2.5 mg tablets packaged in three individual blister compartments to be taken by the patient at the same time. As a further non-limiting example, an individual dose of 5 mg methotrexate and 1 mg folic acid can be packaged together in a single compartment or two individual compartments, where the methotrexate and folic acid are prescribed to be taken together at the same time, but are always packaged in two individual compartments where they are prescribed to be taken at different times. As a further non-limiting example, two individual doses of 0.5 mg methotrexate can each be provided as a single tablet in two individual compartments to be administered twice daily, with 1 mg folic acid packaged together in the same compartment with the first methotrexate dose of the day.

The term “dispenser,” as used herein, means a structure that includes individual compartments, which are means for retaining and physically separating individual doses, or portions therefore. It is understood that a dispenser is amenable to removal of an individual dose and that the individual compartments of a dispenser can open reversibly or irreversibly. In one embodiment, each individual compartment is located at a fixed position relative to the other individual compartments. A dispenser useful in the invention can optionally include, if desired, a visual or audio alarm or a means for recording when individual compartments are opened.

In view of the definition of an individual dose, one skilled in the art understands that each individual compartment in a dispenser contains at most one individual dose and never contains two or more individual doses together; in some cases, an individual compartment contains only a portion of an individual dose, such as one 2.5 mg methotrexate tablet where the total individual dose is 7.5 mg.

A variety of dispensers are useful in a compliance system of the invention including, without limitation, a blister pack composed of, for example, disposable cardboard or paper or a reusable plastic card; a surface with doses of medicament removably affixed thereto; a substantially circular dispenser with compartments for every day of the month; a dispenser containing pre-determined dose injection units; or a credit-card style medication package containing a month's worth of medication. Dispensers known in the art and suitable for use in the compliance systems of the invention further include, but are not limited to, those described in U.S. Pat. No. 4,736,849; U.S. Pat. No. 4,889,236; U.S. Pat. No. 5,265,728; U.S. Pat. No. 6,039,208; U.S. Pat. No. 6,138,866; U.S. Pat. No. 6,439,422; GB 2 237 204 A; publication 0 393 942 A1; and WO 01/68454 A2. Commercially available dispensers also are useful in the invention such as, without limitation, SlidePack®, E-Ztear (PCI Services, Inc.; Cardinal Health), Pill Pak™, and DialPak® tablet dispensers (Ortho Pharmaceutical Corporation; Raritan, N.J.). One skilled in the art understands that these and other disposable or refillable dispensers, including electronic dispensers and those with audio or visual cues, can be useful in the compliance systems of the invention.

A compliance system of the invention can include a plurality of dispensers. As non-limiting examples, a compliance system can include one, two, three, four, five, six, seven, eight, nine, ten, eleven or twelve dispensers each containing a complete weekly or monthly dose of anti-folate therapeutic and folic acid analog. Where multiple dispensers are packaged together in a compliance system, the dispensers can be of the same or different types, and further can be of the same type containing identical or different individual doses of anti-folate therapeutic or folic acid analog.

In one embodiment, a compliance system of the invention includes exactly four dispensers, each including exactly one weekly dose of anti-folate therapeutic and exactly one weekly dose of folic acid analog packaged in the compliance system as at least one individual dose of the anti-folate therapeutic and at least five daily individual doses of the folic acid analog, where each individual dose of anti-folate therapeutic or folic acid analog is positioned in one or more individual compartments and where each dispenser has suitable indicia marked in association with each individual compartment, thereby identifying each compartment with the day or time when the enclosed anti-folate therapeutic or folic acid analog should be administered.

A blister pack is a dispenser which can be useful in the compliance systems of the invention. As used herein, the term “blister pack” is a dispenser in which each compartment is an individual cavity having a rupturable backing. In one embodiment, the cavities or “pockets” are translucent. In a blister pack, individual doses of medication are dispensed by pushing the medication through the rupturable backing.

In one embodiment, a blister pack dispenser useful in the invention includes a first sheet having a plurality of apertures, each aperture defining an opening having an area large enough for the individual dose of anti-folate therapeutic or folic acid analog to pass through; and a second sheet overlapping a portion of the first sheet, said second sheet forming a plurality of hollow cavities, said hollow cavities sealed with a rupturable backing to form a plurality of blister compartments arranged in a pattern on the sheet, with each rupturable backing arranged to overlap each aperture.

The first sheet generally is made of a moderately rigid material such as cardboard or coated cardboard, or plastic such as, without limitation, polyvinyl chloride of a thickness of about 0.5 mm to about 1 mm. The apertures can be of a variety of shapes, for example, circular, elliptical or of another shape appropriate to the egress of the anti-folate therapeutic or folic acid analog. The second sheet is typically formed of a thin, flexible material such as clear polyvinyl chloride or other flexible material including, but not limited to, other translucent materials. The hollow cavities can be formed, for example, by thermal vacuum-drawing of the second sheet in accordance with standard practices in the packaging art. The rupturable backing can be formed, for example, of a thin layer of any of a variety of frangible materials such as metal foil. As one example, aluminum foil, of a thickness of between about 0.25 mm to 0.15 mm can be used as the rupturable backing. In one embodiment, a blister pack is of a size that can be conveniently accommodated in a shirt or other pocket. Such a blister pack can be, for example, of a size of 3 to 4 inches by 4 to 5 inches. A variety of blister packs are known in the art and have been described hereinabove.

As another non-limiting example, a dispenser can be a DialPak® tablet dispenser in which tablets are arrayed circularly and rotated one at a time to an aperture through which a selected tablet can be expelled from the package, with days of the week provided as indicia to guide the user to the appropriate tablet for the current day.

A dispenser useful in the invention optionally includes an audio or visual alarm or means for electronic compliance monitoring, or both; such dispensers are well known in the art and encompass, without limitation, U.S. Pat. No. 4,617,557; U.S. Pat. No. 5,289,157; U.S. Pat. No. 5,852,408; U.S. Pat. No. 6,401,991; WO 02/083057 A1; as well as the Med-ic™m ECM™ available from Information Mediary Corporation (Ontario, Canada). An audio or visual alarm also can be included in a compliance system of the invention in a form separate from the dispenser. Non-limiting examples of separate audio or visual alarms include electronic messaging watches and programmed medication clocks such those described in U.S. Pat. No. 6,075,755 or U.S. Pat. No. 4,837,719, respectively.

In one embodiment, a dispenser useful in the invention includes a means for recording when individual compartments are opened. As an example, a blister pack dispenser can include a means for recording when individual compartments are opened; when the patient dispenses an individual dose from a given compartment, an electronic signal recording the time the compartment was opened is stored. This information can be downloaded to a physician's or pharmacist's computer to track patient compliance, or can be observed real-time, where the dispenser is integrated with a computer system. Means for electronic compliance monitoring are known in the art and include Med-ic™m ECM™M and additional systems described hereinabove.

A dispenser useful in a compliance system of the invention can be optionally marked with suitable indicia in association with each compartment. Such indicia can be, for example, the days of the week or the month or abbreviations therefore such as “M” “T” “W” “Th” “F” “S” “S” or, for example, “Day 1,” “Day 2,” etc. through “Day 7 or “Day 1,” “Day 2,” etc. through “Day 31.” Suitable indicia also can include, for example, the time of day such as, without limitation “morning” and “evening;” “breakfast” and “dinner;” “lunch” and “bedtime,” “breakfast,” “lunch,” “dinner” and “bedtime;” or “A.M.” and “P.M.” In some cases, the indicia may apply to multiple compartments. As one example, a bracket or equivalent symbol can be used to indicate the same dose of folic acid analog included in multiple compartments. As a further example, in a calendar pack containing a month's worth of medication, the designation “Monday” placed above a column of four compartments can refer to each of the four compartments.

In particular embodiments, multiple dispensers are included in a compliance system of the invention, with each dispenser containing exactly a week's worth of medication divided into individual doses. As a non-limiting example, four dispensers can be included in a compliance system of the invention, with each dispenser including exactly a week's worth of medication (weekly dose of anti-folate therapeutic and weekly dose of folic acid analog). In cases in which a compliance system contains multiple dispensers, each separate dispenser can be optionally marked with “Week 1,” “Week 2,” “Week 3,” “Week 4” etc. Alternatively, each dispenser can be optionally marked with “1,” “2,” “3,” and “4” or “Dispenser 1,” “Dispenser 2,” “Dispenser 3,” and “Dispenser 4” or other equivalent language. It is understood that multiple dispensers included together in a compliance system of the invention may not be marked so as to be distinguishable from each other.

The suitable indicia marked in association with each compartment can include, if desired, the name of the anti-folate therapeutic or name of the folic acid analog or appropriate abbreviation. As another option, the dose of one or both of the anti-folate therapeutic and folic acid analog also can be marked on the dispenser in association with the appropriate compartment.

All of the elements of a compliance system of the invention can be optionally packaged in an outer container made of any suitable material. Such an outer container can be constructed, for example, of any appropriate paper or plastic material, or a combination thereof. The outer container typically is of a size to accommodate standard pharmacy prescription labels and can have, without limitation, a rectangular or square shape.

It further is understood that a compliance system of the invention, with or without an outer container, can be packaged in a child-resistant manner or tamper-evident manner or both. Child-resistant blister packages can incorporate, for example, at least one of the child-resistant features described in ASTM D-3475, or another feature which meets standard requirements for child resistance. Well known child-resistant blister cards included SlidePack® and E-Ztear packages. Additional child-resistant packaging, including child-resistant blister packaging, also is well known in the art, as described, for example, in U.S. Pat. Nos. 3,503,493; 3,809,220; 3,809,221; 3,924,746; 3,924,747; 4,011,949; 4,398,634; and 4,537,312. One skilled in the art understands that these and other child-resistant and tamper-evident dispensers and outer containers can be useful in a compliance system of the invention.

A compliance system of the invention optionally includes one or more reminder aids. Such a reminder aid can be, without limitation, one or any combination of reminder cards with information to remind the patient when to take a dose of medication; adhesive stickers with information to remind the patient when to take a dose of medication; or a visual or audio alarm that is activated at the time an individual dose should be taken. It is understood that visual and audio alarms encompass those to be set by the patient as well as those set, for example, by the manufacturer or pharmacist. Audio alarms useful in the invention encompass, without limitation, buzzes, beeps, music and verbal cues such as “Time to take methotrexate.” Visual cues useful in the invention include, yet are not limited to, a light that turns from off to on; a light that turns from a first color such as red to a second color such as green; or a light that begins flashing at the time when an individual dose has been prescribed to be taken. A variety of dispensers which include or can be used in conjunction with an audio or visual alarm such as a watch or a clock are known in the art and have been described hereinabove. One skilled in the art understands that these and a variety of other audio, visual or other cues can be useful in the compliance systems of the invention.

A compliance system of the invention further optionally includes patient information provided separately from any outer container and the one or more dispensers. Such patient information can be provided, for example, as a paper insert or booklet and generally includes dosing information. The patient information provided can further optionally include, without limitation, side effect information, patient incentive information or information on the disease being treated. The term “patient information,” as used herein, means any information of interest to a patient being treated with an anti-folate therapeutic. Such patient information includes, but is not limited to, any or all of the following: dosage information; importance of complying with dosage and administration instructions; side effect information, optionally including when during therapy side effects typically occur or how to manage side effects; anticipated benefits of therapy; and information regarding the disease or condition being treated. The patient information can additionally include, if desired, instructions regarding how and when to make up any missed doses as well as patient incentive information such as statements that encourage compliance by highlighting the benefits of proper administration. The information is generally provided in a form which avoids complex and difficult medical terminology, using simple words appropriate to all educational levels.

Patient information relating to methotrexate can include information regarding early signs of side effects. Patients may be informed to notify their doctor immediately if any of the following are observed: an allergic reaction (shortness of breath, closing of the throat or difficulty breathing); fever or chills; a sore throat, unusual bruising or bleeding; unexplained shortness of breath, coughing or wheezing; diarrhea; abdominal pain; black, tarry stools; sores in or around the mouth; yellow skin or eyes; blood in the urine; darkened urine; swelling of the feet or legs; joint pain; or confusion, unusual behavior or seizures. Patient information also can include information regarding less serious side effects sometimes caused by methotrexate such as nausea, vomiting or decreased appetite; itching or skin rash; hair loss; boils or acne; dizziness; increased sensitivity of skin to sunlight; headache; drowsiness; or blurred vision. Patients can be informed to continue taking methotrexate while notifying their doctor of any such side effects and can be informed that they should contact their doctor regarding any usual or especially bothersome side effect.

Patient information also can include, for example, warnings regarding possible drug interactions with methotrexate. Patients can be informed, for example, to talk with a doctor before taking aspirin or nonsteroidal anti-inflammatory drugs (NSAIDs) such as ibuprofen (Advil, Motrin or Nuprin), ketoprofen (Orudis KT, Orudis, Oruvail), or naproxen (Aleve, Naprosyn, Anaprox), indomethacin (Indocin), oxaprozin (Daypro), sulindac (Clinoril), tolmetin (Tolectin), ketorolac (Toradol), diclofenac (Cataflam, Voltaren), etodolac (Lodine), fenoprofen (Nalfon), flurbiprofen (Ansaid), nabumetone (Relafen), or piroxican (Feldene). Patients can be informed that such medications could interact with methotrexate and result in death.

Patient information also can include an indication to discuss the following medications with a doctor: Etretinate (Tegison); theophylline (Theo-Dur, Theolair, Theochron, Elixophyllin, Slo-Phyllin, others); phenyloin (Dilantin); probenecid (Benemid); procarbazine (Matulane); folic acid or a vitamin supplement that contains folic acid; a penicillin antibiotic such as ampicillin (Principen, others), amoxicillin (Amoxil, Trimoz, Augmentin, others), dicloxacillin (Dynapen, others), penicillin (Pen-Vee-K, Veetids, others), and others; a tetracycline antibiotic such as minocycline (Minocin, Dynacin, others), doxycycline (Vibramycin, Vibra-Tabs, others), tetracycline (Sumycin, others), and others; or a sulfa-based medicine such as sulfamethoxazole (Bactrim, Septra, Sulfatrim, Gantanol), sulfisoxazole (Gantrisin), and others. Patients can be informed that a dosage adjustment or special monitoring may be necessary. Patient information can further include an indication that other drugs also can interact with methotrexate and that patients should discuss any prescription or over-the-counter medication with a doctor. Patient information also can include a warning to avoid alcohol and a warning to avoid prolonged exposure to sunlight due to increased skin sensitivity.

Patient information also can include information regarding conditions that may be inconsistent with methotrexate usage or which may require a special dosage or special monitoring such as, without limitation, liver disease or a history of liver problems; kidney disease; alcoholism or alcoholic liver disease; immune disorders; infections; stomach ulcers; ulcerative colitis; diabetes; fluid around the lungs or abdomen; blood problems; or asthma, chronic obstructive pulmonary disease (COPD), chronic bronchitis, or any other lung disease. Patient information also can include warnings regarding taking methotrexate prior to conception, during pregnancy or while breast feeding.

Additional patient information can include contraindications for taking folinic acid such as hypersensitivity to leucovorin, pernicious anemia or other megaloblastic anemias where a deficiency of B12 is present. Patient information also can include warnings of adverse reactions to folinic acid such as seizures, thrombocytosis, wheezing and anaphylactoid reactions.

The compliance systems of the invention include both an anti-folate therapeutic and a folic acid analog. For convenience, the term “medicament” is used herein to mean either an anti-folate therapeutic or a folic acid analog, each of which is described further below.

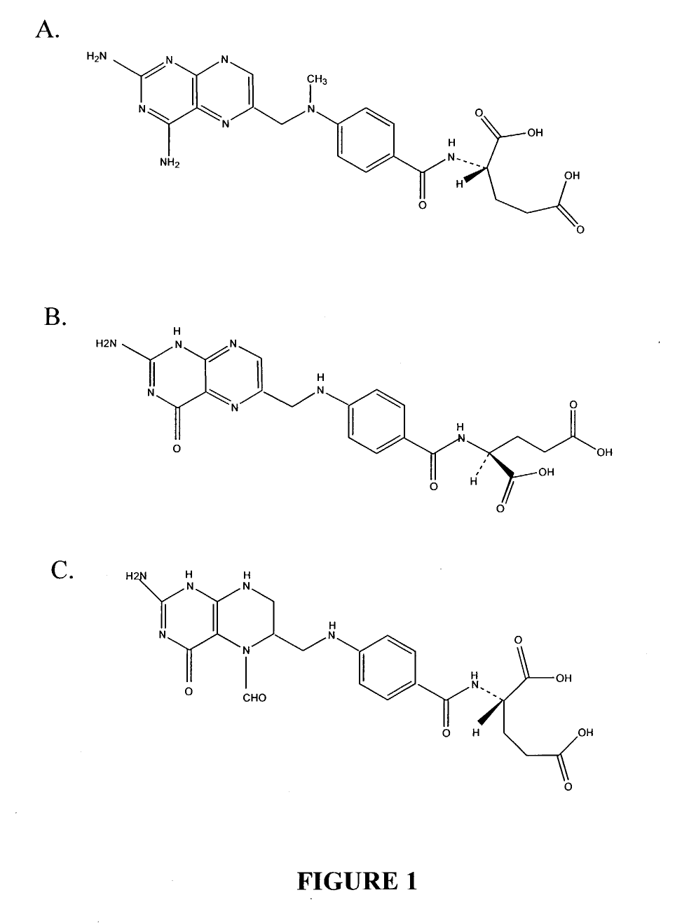

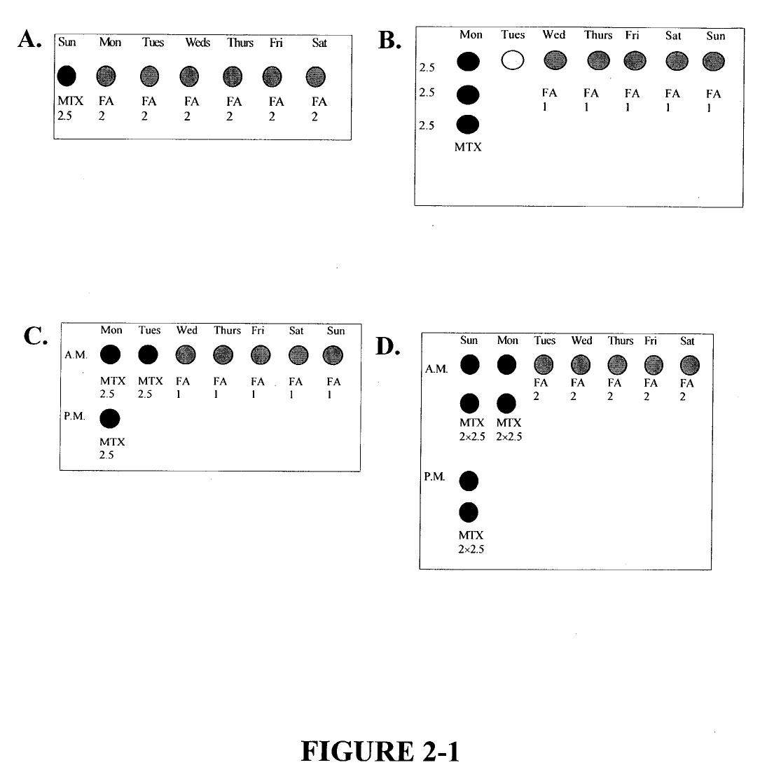

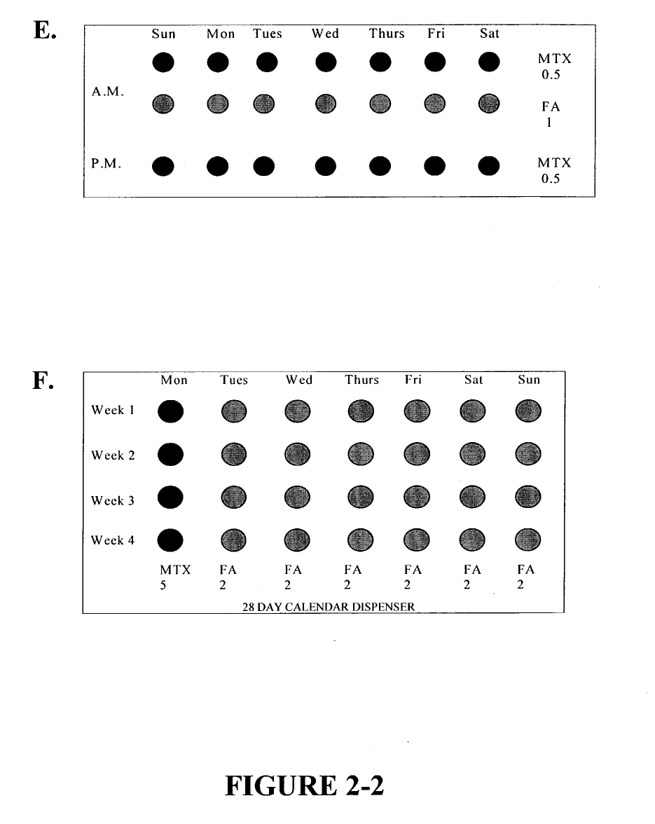

An anti-folate therapeutic useful in the invention can be, without limitation, methotrexate or an analog thereof. As used herein, the term “methotrexate” is synonymous with “MTX” and means a molecule having the structure shown in FIG. 1A. As shown in this figure, methotrexate includes, in part, a 2,4-diamino substituted pterine ring moiety linked at the 6 position to the amino group of a p-aminobenzoyl moiety, the p-aminobenzoyl moiety having a methylated amino group and linked to a glutamic acid moiety through an amide bond. Methotrexate functions as an inhibitor of dihydrofolate reductase (DHFR), decreasing the production of tetrahydrofolate (THF) from dihydrofolate (DHF). As a consequence, methotrexate indirectly inhibits purine and thymidine synthesis and amino acid interconversion. Methotrexate also exhibits anti-proliferative activity through inhibition of thymidylate synthesis, which is required for production of DNA (Calvert, Semin. Oncol. 26:3-10 (1999)).

Methotrexate is administered orally or parenterally and is readily distributed to body tissues, where it is transported into cells by a specific carrier system which includes components such as the reduced folate carrier, RCF1, and the folate receptor. Due to its high polarity at physiological pH, methotrexate does not readily pass through the cell membrane, and the majority of methotrexate enters cells via specific carriers. Once inside the cell, methotrexate is converted to methotrexate polyglutamates by specific enzymes such as folylpoly-gamma-glutamate synthetase, which add one or more glutamic acid moieties to the γ-carboxyl of methotrexate (Kamen, Semin. Oncol. S18:30-39 (1997)). Methotrexate and its synthesis and properties are described in further detail in U.S. Pat. Nos. 2,512,572; 3,892,801; 3,989,703; 4,057,548; 4,067,867; 4,079,056; 4,080,325; 4,136,101; 4,224,446; 4,306,064; 4,374,987; 4,421,913; 4,767,859; 4,106,488; 4,558,690; and 4,662,359. Methotrexate is commercially available, for example, under the name Rheumatrex from Lederle Laboratories.

Pharmaceutically acceptable derivatives of methotrexate also can be useful in the invention including, without limitation, salts, esters, amides, sterioisomers, racemic mixtures, polymorphs, hydrates and solvates. Such pharmaceutically acceptable derivatives can have substantially the activity of methotrexate in antagonizing dihydrofolate reductase.

Pharmaceutically acceptable salts of methotrexate include, without limitation, acid addition salts, which can be formed using a solution of an appropriate acid such as hydrochloric acid, sulfuric acid, fumaric acid, maleic acid, succinic acid, acetic acid, benzoic acid, citric acid, tartaric acid, carbonic acid or phosphoric acid. Pharmaceutically acceptable salts further include, yet are not limited to, acid phosphate, acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroiodide, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium, oleate, oxalate, pamoate (embonate), palmitate, pantothenate, phosphate/diphosphate, polygalacturonate, saccharate, salicylate, stearate, sulfate, subacetate, succinate, tannate, tartrate, teoclate, p-toluene sulphonate salts, tosylate, triethiodide and valerate.

It further is understood that functional groups of methotrexate can be modified, for example, to enhance the pharmacological utility of the compound. Such modifications are well within the knowledge of the skilled chemist and include, without limitation, esters, amides, ethers, N-oxides, and pro-drugs of methotrexate, which are encompassed within the term “methotrexate” as used herein. Examples of modifications that can enhance activity include, for example, esterification such as the formation of C₁ to C₆ alkyl esters, preferably C₁ to C₄ alkyl esters, wherein the alkyl group is a straight or branched chain. Other acceptable esters include, for example, C₅ to C₇ cycloalkyl esters and arylalkyl esters such as benzyl esters. Such esters can be prepared from the compounds described herein using conventional methods well known in the art of organic chemistry.

Other pharmaceutically acceptable modifications include the formation of amides. Useful amide modifications include, for example, those derived from ammonia; primary C₁ to C₆ dialkyl amines, where the alkyl groups are straight or branched chain; and arylamines having various substitutions. In the case of secondary amines, the amine also can be in the form of a 5 or 6 membered ring. Methods for preparing these and other amides are well known in the art.

It is further understood that chemically distinct enantiomers and tautomers of methotrexate can be useful in the compliance systems of the invention. Furthermore, in crystalline form, a compound may exist as polymorphs; in the presence of a solvent, a compound may form a solvate, for example, with water or a common organic solvent. Such polymorphs, hydrates and other solvates of methotrexate also are encompassed within the term “methotrexate” and can be useful in the compliance systems of the invention disclosed herein.

A compliance system of the invention includes methotrexate or another anti-folate therapeutic. As used herein, the term “anti-folate therapeutic” means a molecule having structural similarity to folate and activity as a folate antagonist against one or more folate-dependent enzymes. The term anti-folate therapeutic, which encompasses molecules known as folic acid antagonists or folate analogs, includes, for example, polyglutamylatable drugs with folate antagonist activity and further encompasses, without limitation, aminopterin, trimetrexate, edatrexate, raltitrexed (Tomudex®), lometrexol, multitargeted antifolate (MTA), AQA, 1843U89 and analogs thereof (Longo-Sorbello and Bertino, Haematol. 86:121-127 (2001)). Aminopterin, for example, possesses a hydrogen instead of a methyl group at position N-10 compared to the structure of methotrexate. Raltitrexed is a selective inhibitor of thymidylate synthase as described, for example, in Kamen, Semin. Oncol. S18:30-39 (1997). Lometrexol selectively inhibits glycinamide ribonucleotide formyltransferase, the first enzyme involved in the pathway of de novo purine synthesis as described, for example, in Calvert, supra, 1999. Multitargeted antifolate (MTA; LY231514) is an inhibitor of multiple folate-dependent enzymes, such as dihydrofolate reductase, thymidylate synthase, and glycinamide ribonucleotide formyltransferase, and is an efficient substrate for polyglutamation (Calvert, supra, 1999; Mendelsohn et al., Sem. Oncol. 26 (Suppl. 6): 42-47 (1999); and Rinaldi, Sem. Oncol. 26 (Suppl. 6): 82-88 (1999)). These and other anti-folate therapeutics with reduced, equivalent or improved antagonist activity as compared to methotrexate against one or more folate-dependent enzymes can be useful in a compliance system of the invention.

The term anti-folate therapeutic further includes, but is not limited to, compounds with increased lipid solubility, membrane transport or ability to form long chain polyglutamates relative to methotrexate (Longo-Sorbello and Bertino, supra, 2001). As a non-limiting examples, an anti-folate therapeutic can be trimetrexate (TMTX) or an analog thereof. Trimetrexate is a non-classic, quinazoline-derived lipophilic anti-folate that achieves high intracellular concentrations in cells that are resistant to methotrexate due to defective transport thereof (Fleisher, Ther. Drug Monit. 15:521-526 (1993); Kheradpour et al., Cancer Invest. 13:36-40 (1995); and Gangjee et al., J. Med. Chem. 40:479-485 (1997)). An anti-folate therapeutic also can be edatrexate (EDX), or an analog thereof such as an N10-propargyl analog, which is structurally similar to methotrexate with a higher therapeutic index (Sirotnak et al., Cancer Chemother. Pharm. 12:26-30 (1984); and Mauritz et al., Clin. Cancer Res. 4:2399-2410 (1998)). Additional anti-folate therapeutics include inhibitors of thymidylate synthetase such as Raltitrexed, which enters cells via active transport and is a substrate for polyglutamylation by FPGS (Jackman et al., Cancer Res. 51:5579-5586 (1991)). One skilled in the art understands that these and other drugs with structural similarity to folate and activity as a folate antagonist against one or more folate-dependent enzymes are encompassed by the term “anti-folate therapeutic” and are useful in the compliance systems of the invention.

One type of anti-folate therapeutic useful in the invention is a methotrexate analog. As used herein, the term “methotrexate analog” means a molecule with structural similarity to methotrexate, which functions to inhibit the enzyme dihydrofolate reductase. A methotrexate analog useful in the invention acts as a substrate for polyglutamylation in a cell by an enzyme such as folylpoly-gamma-glutamate synthetase. Methotrexate analogs include, but are not limited to, 4-amino derivatives with halogen substitution on the para-aminobenzoic moiety, such as dichloromethotrexate (Frei et al., Clin. Pharmacol. Therap. 6:160-71 (1965)); 7-methyl substituted methotrexate (Rosowsky and Chen, J. Med. Chem. 17:1308-11 (1974)); 3′,5′-difluoro methotrexate (Tomcuf, J. Organic Chem. 26:3351 (1961)); 2′ and 3′ monofluorinated derivatives of aminopterin (Henkin and Washtien, J. Med. Chem. 26:1193-1196 (1983)); and 7,8-dihydro-8-methyl-methotrexate (Chaykovsky, J. Orq. Chem. 40:145-146 (1975)). The skilled person understands that a compliance system of the invention can incorporate methotrexate analogs or other anti-folate therapeutics, or a combination thereof, in the same manner as disclosed herein for methotrexate.

In addition to an anti-folate therapeutic, a compliance system of the invention includes a folic acid analog. The term “folic acid,” as used herein, means pteroylglutamic acid as shown in FIG. 1B or a derivative having a different level of reduction of the bicyclic pteridine portion, one or more substitutions of the pteridine portion, or a different number of glutamate residues. As shown in FIG. 1B, folic acid is composed of glutamic acid, p-aminobenzoic acid and pteridine derivative components. Folic acid itself has no coenzyme activity but is enzymatically reduced to dihydrofolic acid and subsequently to tetrahydrofolic acid. Folic acid can be synthesized, for example, as described in Angier et al., Science 103:667 (1946)).

The term “folinic acid,” as used herein, is synonymous with leucovorin, citrovorum factor and 5-formyl tetrahydrofolate and means the 5-formyl derivative of tetrahydrofolic acid shown in FIG. 1C. Folinic acid, a reduced form of folic acid, does not require reduction for activation and is commercially available, for example, as Leucovorin calcium, for example, in the form of 5 mg tablets, from Barr Laboratories (Pomona; NY); Par Laboratories, Inc. (Charlotte, N.C.); Roxane Laboratories, Inc. (Columbus, Ohio); PCH Pharmachemie (The Netherlands); and Lederle Laboratories (Madison, N.J.) and under the name Wellcovorin®. Folinic acid also can be prepared by routine synthetic methods, as described, for example, in U.S. Pat. No. 5,350,851.

As used herein, the term “folic acid analog” means a compound structurally similar to folic acid in its reduced or oxidized form, or a precursor thereto, and being metabolized to act as a coenzyme for one or more folate-dependent enzymes such as dihydrofolate reductase or thymidylate synthetase. Thus, the term “folic acid analog” encompasses folic acid as well as folinic acid and structurally similar compounds that can substitute for folic acid in alleviating side effects resulting from folate deficiency, for example, side effects resulting from methotrexate administration. The term folic acid analog encompasses, without limitation, folic acid, dihydrofolic acid, tetrahydrofolic acid, 5-formyl-tetrahydrofolic acid and 10-methyl-tetrahydrofolic acid.

A compliance system of the invention can optionally include any of a variety of drugs or other active compounds in addition to the anti-folate therapeutic and folic acid analog. Such a drug or active compound can be, for example, any drug or compound beneficial to an individual having, for example, rheumatoid arthritis, psoriasis, Crohn's disease, ulcerative colitis, systemic lupus erythematosus, systemic vasculitis, polymiositis, multiple sclerosis, Type 1 diabetes, autoimmune uveoretinitis, myasthenia gravis, autoimmune thyroiditis, asthma or graft-versus-host disease. Such a drug or active compound can be, without limitation, an anti-metabolite, corticosteroid or other anti-inflammatory agent, immunosuppressant, immunomodulating drug, anti-cytokine agent, drug or active compound that synergizes with methotrexate, or drug or supplement that reduces one or more side effects associated with methotrexate or another component of the compliance package. As non-limiting examples, an additional drug or active component in a compliance system of the invention can be glutamine; sulfasalazine or another 5-aminosalicylate; an anti-metabolite such as leflunomide (Arava®), azathioprine, 6-mercaptopurine or 6-thioguanine; a corticosteroid such as prednisone or prednisolone; an immunosuppressant such as cyclophosphamide (for example, Cytoxan® or Neosar®), cyclosporine (for example, Neoral® or Sandimmune®), hydroxychloroquine (for example, Plaquenil®), azathioprine (for example, Imuran®), 6-mercaptopurine (6-MP) or 6-thioguanine (6-TG); an anti-cytokine therapeutic, for example, an anti-TNF-α antibody or other anti-TNF-α therapeutic such as ENBREL® or Remicade® or an anti-IL-1 therapeutic such as an IL-1 receptor antagonist (IL-1Ra), for example, Anakinra®; or a p38 kinase inhibitor. It is further understood that an anti-folate therapeutic or a folic acid analog can be formulated together with one or more additional active ingredients. As an example, folic or folinic acid can be provided in the form of a multi-vitamin.

A compliance system of the invention can optionally include one or more placebos. A placebo lacks folate agonist or antagonist activity and generally is any substance lacking significant pharmacological activity. In one embodiment, a compliance system of the invention includes a placebo for every day on which no anti-folate therapeutic or folic acid analog is prescribed. As a non-limiting example, a compliance system of the invention can include one to four dispensers, each dispenser having one individual dose of anti-folate therapeutic, five daily individual doses of a folic acid analog to be given on days when the anti-folate therapeutic is not prescribed, and one individual dose of a placebo to be given on the day when neither anti-folate therapeutic nor folic acid analog is prescribed.

Anti-folate therapeutics and folic acid analogs included in a compliance system of the invention can be conveniently formulated for oral administration. Anti-folate therapeutics and folic acid analogs also can be formulated for other routes of administration, depending, for example, on the type and severity of condition to be treated, and the history, risk factors and symptoms of the subject. As non-limiting examples, anti-folate therapeutics and folic acid analogs useful in the invention can be formulated for systemic or local administration and further can be formulated for oral administration or for administration by dermal patch; topical drops, creams, gels or ointments; or for parenteral administration; for subcutaneous, intramuscular, intravenous or other injection; and as extended release formulations. Acceptable dosage forms include, without limitation, tablets, pills, capsules, GelCaps (gelatin-coated capsules) and other solid formulations, gels, creams, ointments, suppositories, powders, liquids, suspensions, emulsions, pre-filled syringes, aerosols and the like.

In one embodiment, both the anti-folate therapeutic and the folic acid analog are formulated for oral administration. In further embodiments, the anti-folate therapeutic and the folic acid analog are supplied in in the same or different type of solid formulation such as, without limitation, tablets, pills, capsules or GelCaps.

Pharmaceutical compositions containing methotrexate or another anti-folate therapeutic, as well as pharmaceutical compositions containing a folic acid analog such as leucovorin optionally include an excipient such as a pharmaceutically acceptable carrier or a diluent, which is any carrier or diluent that has substantially no long term or permanent detrimental effect when administered to a human. An excipient generally is mixed with active compound, or permitted to dilute or enclose the active compound. A carrier can be a solid, semi-solid, or liquid agent that acts as an excipient or vehicle for the active compound. Examples of solid carriers include, without limitation, pharmaceutical grades of mannitol, lactose, starch, magnesium stearate, sodium saccharin, polyalkylene glycols, talcum, cellulose, glucose, sucrose and magnesium carbonate. Suppository formulations can include, for example, propylene glycol as a carrier. Examples of pharmaceutically acceptable carriers and diluents include, without limitation, water, such as distilled or deionized water; saline; aqueous dextrose, glycerol, ethanol and the like. It is understood that the active ingredients can be soluble or can be delivered as a suspension in the desired carrier or diluent.

A pharmaceutical composition including the anti-folate therapeutic or folic acid analog also can optionally include one or more agents such as, without limitation, emulsifying agents, sweetening or flavoring agents, tonicity adjusters, preservatives, buffers, anti-oxidants or wetting agents. Tonicity adjustors useful in a pharmaceutical composition include, but are not limited to, salts such as sodium acetate, sodium chloride, potassium chloride, mannitol or glycerin and other pharmaceutically acceptable tonicity adjustors. Preservatives useful in pharmaceutical compositions include, without limitation, benzalkonium chloride, chlorobutanol, thimerosal, phenylmercuric acetate, and phenylmercuric nitrate. Various buffers and means for adjusting pH can be used to prepare a pharmaceutical composition, including, but not limited to, acetate buffers, citrate buffers, phosphate buffers and borate buffers. Similarly, anti-oxidants useful in pharmaceutical compositions are well known in the art and include, for example, sodium metabisulfite, sodium thiosulfate, acetylcysteine, butylated hydroxyanisole and butylated hydroxytoluene. It is understood that these and other substances known in the art of pharmacology can be included in a pharmaceutical composition containing an anti-folate therapeutic or a folic acid analog in a compliance system of the invention. See, for example, Remington's Pharmaceutical Sciences Mack Publishing Company, Easton, Pa. 16^{th} Edition 1980.

All journal article, reference and patent citations provided above, in parentheses or otherwise, whether previously stated or not, are incorporated herein by reference in their entirety.

Although the invention has been described with reference to the examples provided above, it should be understood that various modifications can be made without departing from the spirit of the invention. Accordingly, the invention is limited only by the claims.

We claim:

1. A compliance system, comprising at least one dispenser comprising at least one individual dose of an anti-folate therapeutic and at least one individual dose of a folic acid analog, each individual dose of anti-folate therapeutic and each individual dose of folic acid analog positioned in one or more individual compartments.

2. The compliance system of claim 1, said dispenser having suitable indicia marked in association with each individual compartment, thereby identifying each compartment with the day or time when the enclosed anti-folate therapeutic or folic acid analog should be administered.

3. The compliance system of claim 1, said dispenser comprising at least one individual dose of said anti-folate therapeutic and at least five daily individual doses of said folic acid analog.

4. The compliance system of claim 3, wherein said dispenser comprises at least one individual dose of said anti-folate therapeutic and exactly six daily individual doses of said folic acid analog.

5. The compliance system of claim 1, 3 or 4, wherein said anti-folate therapeutic is methotrexate.

6. The compliance system of claim 1, 3 or 4, wherein each dispenser has exactly one weekly dose of anti-folate therapeutic.

7. The compliance system of claim 1, 3 or 4, wherein each dispenser has exactly one weekly dose of methotrexate.

8. The compliance system of claim 7, wherein said weekly dose of methotrexate is from 2.5 mg to 40 mg.

9. The compliance system of claim 8, wherein said weekly dose of methotrexate is selected from the group 2.5 mg, 5 mg, 7.5 mg, 10 mg, 15 mg, 20 mg and 25 mg.

10. The compliance system of claim 9, wherein said weekly dose of methotrexate is 2.5 mg.

11. The compliance system of claim 10, wherein said weekly dose of 2.5 mg methotrexate is a single individual dose.

12. The compliance system of claim 9, wherein said weekly dose of methotrexate is 5 mg.

13. The compliance system of claim 12, wherein said weekly dose of 5 mg methotrexate is a single individual dose.

14. The compliance system of claim 9, wherein said weekly dose of methotrexate is 7.5 mg.

15. The compliance system of claim 14, wherein said weekly dose of 7.5 mg methotrexate is a single individual dose.

16. The compliance system of claim 14, wherein said weekly dose of 7.5 mg methotrexate is separated into two or more individual doses.

17. The compliance system of claim 9, wherein said weekly dose of methotrexate is 10 mg.

18. The compliance system of claim 17, wherein said weekly dose of 10 mg methotrexate is a single individual dose.

19. The compliance system of claim 17, wherein said weekly dose of 10 mg is separated into two or more individual doses.

20. The compliance system of claim 9, wherein said weekly dose of methotrexate is 15 mg.

21. The compliance system of claim 20, wherein said weekly dose of 15 mg methotrexate is a single individual dose.

22. The compliance system of claim 20, wherein said weekly dose of 15 mg methotrexate is separated into two or more individual doses.

23. The compliance system of claim 1, 3 or 4, wherein said folic acid analog is folic acid.

24. The compliance system of claim 1, 3 or 4, wherein said folic acid analog is folinic acid.

25. The compliance system of claim 24, wherein said daily individual dose of folinic is from 0.5 to 2 mg.

26. The compliance system of claim 25, wherein said daily individual dose of folinic acid is 1 mg or 2 mg.

27. The compliance system of claim 1, 3 or 4, wherein said individual compartments are arranged linearly.

28. The compliance system of claim 1, 3 or 4, comprising at least two of said dispensers.

29. The compliance system of claim 1, 3 or 4, comprising four of said dispensers.

30. The compliance system of claim 1, 3 or 4, further comprising a patient information insert.

31. The compliance system of claim 1, 3 or 4, further comprising an outer container.

32. The compliance system of claim 1, 3 or 4, wherein said at least one dispenser further comprises a visual or audio alarm.

33. The compliance system of claim 1, 3 or 4, wherein said at least one dispenser further comprises means for recording when individual compartments are opened.

34. A compliance system, comprising four dispensers,
each dispenser comprising at least one individual dose of an anti-folate therapeutic and at least five daily individual doses of a folic acid analog, each individual dose of anti-folate therapeutic and each individual dose of folic acid analog positioned in one or more individual compartments,
each dispenser having suitable indicia marked in association with each individual compartment, thereby identifying each compartment with the day or time when the enclosed anti-folate therapeutic or folic acid analog should be administered.

35. A compliance system, comprising at least one dispenser, said dispenser comprising exactly four weekly doses of anti-folate therapeutic and exactly four weekly doses of folic acid analog,
each of said weekly doses of anti-folate therapeutic or folic acid analog divided into individual doses sequentially arrayed in said dispenser.
